(19) United States
(12) Patent Application Publication
(10) Pub. No.: US 2014/0121770 A1
(43) Pub. Date: May 1, 2014

(54) Method for Adjusting Breast Regeneration Part

(71) Applicants: Noriyuki Ikeyama, Nagoya-shi (JP); Meisei Takeishi, Shizuoka (JP)

(72) Inventors: Noriyuki Ikeyama, Nagoya-shi (JP); Meisei Takeishi, Shizuoka (JP)

(73) Assignee: Ikeyama Medical Japan Co., Ltd., Nagoya-shi, Aichi (JP)

(21) Appl. No.: 14/125,379

(22) PCT Filed: May 31, 2012
(86) PCT No.: PCT/JP2012/064178

§ 371 Date: Dec. 11, 2013

(30) Foreign Application Priority Data
Jun. 16, 2011 (JP) 2011-134293

(51) Int. Cl. A61F 2/12

(52) U.S. Cl. CPC A61F 2/12
USPC 623/8

(57) ABSTRACT

The present invention comprises, when adjusting a breast substitute (10) for the preparation of breast reconstruction; abreast female mold creating step (101) of transcribing the existing breast or the breast to be removed and creating a breast female mold (1); a receiving step (102) of receiving the breast substitute (10) in the concave portion (1a) of the breast female mold (1) so as to include excess part of the concave portion (1a); a size adjusting step or an indicating step of removing the excess part of the concave portion (1a) of the breast substitute (10) as an unwanted part (301) of the breast substitute (10), or indicating the boundary for removal, or folding back the excess part to make it smaller so that an excess part does not form, or indicating a base line for making the excess part smaller; and a substitute preparing step (104) of unloading the breast female mold (1) and preparing as the breast substitute (10) after the size adjusting step or the indicating step. According to the present invention, a method for adjusting a breast reconstruction part is provided for the preparation of achieving good balance of appearance of the breasts after breast reconstruction.

### CROSS REFERENCE TO RELATED APPLICATION

This application is the 35 U.S.C. §371 national stage of PCT application entitled with “Method for adjusting breast reconstruction part” having serial number PCT/JP2012/064178, filed on May 31, 2012. This application also claims priority to and benefit of Japanese Application No. 2011-134293, filed on Jun. 16, 2011, which is incorporated by reference in its entity.

### BACKGROUND OF THE INVENTION

1. Field of the Invention

The invention relates to a method for adjusting abreast regeneration part that adjusts a breast substitute for preparation of breast reconstruction (regeneration).

2. Background Art

Conventionally, when a breast is excised due to breast cancer or other tumors or when one of the breasts is congenitally defectively developed, breast reconstruction has been performed in order to complement balance with the remaining breast for an appearance of the left and right breasts.

As breast reconstruction, a method of embedding an artificial material by a surgical operation and a method of transplanting an autologous tissue by a surgical operation have been known. The Patent Document 1 relates to a breast correction pad after a breast cancer surgery.

### PRIOR ART DOCUMENT

### Patent Document

[Patent Document 1] Japanese Examined Utility Model Application Publication No. 62-23763

### SUMMARY OF THE INVENTION

### Problems to be Solved by the Invention

In this breast reconstruction, when an autologous tissue is used, an autologous tissue that is the original to be transplanted is removed and transplanted into a breast reconstruction part; however, it is difficult to achieve balance (homogenization) between an appearance after a surgery with the autologous tissue that was removed and transplanted and an appearance of the remaining breast.

An object of the invention is to provide a method for adjusting a breast reconstruction part, which enables the preparation for achieving good balance of an appearance of the breasts after breast reconstruction.

### Means for Solving the Problems

In order to solve the above described problem, the method for adjusting a breast regeneration part of the invention includes, when adjusting a breast substitute for the preparation of breast reconstruction; a breast female mold creating step of transcribing the existing breast or the breast to be removed and creating a breast female mold; a receiving step of receiving the breast substitute in a concave portion of the breast female mold so as to include an excess part of the concave portion; a size adjusting step of removing the excess part of the concave portion of the breast substitute as an unwanted part of the breast substitute or folding back the excess part so that an excess part does not form, or an indicating step of indicating the boundary for removing the excess part of the concave portion of the breast substitute as an unwanted part of the breast substitute or indicating a base line for folding back the excess part so that an excess part does not form; and a substitute preparing step of unloading the breast female mold and preparing as the breast substitute after the size adjusting step or the indicating step.

Abreast substitute adjusted by this method is applied to a breast reconstruction part in the chest and the periphery thereof is sutured, and at the same time, for example, when a breast substitute is a living tissue that is an autologous tissue, a blood vessel in the breast substitute and a blood vessel in the chest are connected or the state of continuing the blood vessels is remained, thereby reconstructing the transplanted living tissue as a part of the chest, and thus, the breast is reconstructed. As described above, the invention enables preparation of breast reconstruction, that is, adjustment of a size of a breast substitute.

Conventionally, sizes of the existing breast and the reconstructed breast are apt to be different, and if a part that is supposed to be unwanted in an autologous tissue in the reconstructed side of the breast is excised too much, the size cannot be recovered after that, and therefore, the breast in the reconstructed side tends to inevitably become larger and the left and right breasts are imbalanced in many cases. This is because preparation of a shape and adjustment of a size are carried out by an operator with the eye. In contrast, according to the invention, a shape of the existing breast or the like is previously transcribed into a breast female mold, an autologous tissue excised for reconstruction is received in this breast female mold, and an excess part of the concave portion of the breast female mold, for example, at least one of an excess part formed in the outside and an excess part formed in the upper side, is removed as an unwanted part, or a boundary between the unwanted part and the necessary part is indicated, for example, by marking (then removed), or the excess part of the autologous tissue is folded back to be smaller so that an excess part does not form, a base line for folding back is indicated in the excess part (the size adjusting step or the indicating step for removing thereafter is performed) and, therefore, sizes of the existing breast and the reconstructed breast can be made nearly uniform. Accordingly, since a breast substitute that can be truly reproduced in the shape of the existing breast by the autologous tissue can be prepared, a naturally balanced body shape can be recovered after breast reconstruction, and mental stability can be attained and uneasiness to the social life can be resolved.

In addition, the size adjusting step of the method for adjusting a breast regeneration part of the invention can be an unwanted part removal step of removing an excess part of the concave portion of the breast female mold. Alternatively, the size adjusting step can be a step of reducing a size by folding back and suturing an excess part of the concave portion of the breast female mold. The breast female mold creating step includes a step of building a gelled resin material on the surface of the existing breast or the surface of the breast to be removed, hardening the resin material, and then unloading the resin material from the breast. Due to the step, molding by using the breast as a matrix is facilitated.

In the method for adjusting a breast regeneration part of the invention, skin or a skin flap made of a subcutaneous tissue is favorably used as the autologous tissues.

### BRIEF DESCRIPTION OF THE DRAWINGS

FIG. 1 is a flowchart showing the method for adjusting a breast regeneration part according to the invention.

FIG. 2 is a view showing a breast reconstruction part in the first reconstruction and the second reconstruction.

FIGS. 3A to 3C are views showing a step of creating a breast female mold in the breast female mold creating step.

FIGS. 4A to 4C are views showing a step of creating another example of the same breast female mold above.

FIGS. 5A and 5B are views showing a step of receiving an autologous tissue in the concave portion of the breast female mold.

FIG. 6A is a view showing a state that the autologous tissue received in the concave portion contains an excess part of the breast female mold.

FIG. 6B is a view corresponding to FIG. 6A, which shows an example of retaining the breast female mold in a holder base.

FIG. 7 is a view showing a state of cutting an unwanted part of a living tissue with a knife.

FIG. 8 is a view showing a position that indicates a mark for recognizing an unwanted excess part of a breast female mold in the living tissue.

FIG. 9 is a view showing an indicated mark on a plan view.

FIG. 10 is a view showing another example of the unwanted part removal step.

FIG. 11 is a view showing another example of the unwanted part removal step.

FIG. 12 is a view showing a step of adjusting a shape of a breast substitute by suturing together the autologous tissue.

FIG. 13 is a view showing a step of adjusting a shape of a breast substitute by removing an unwanted part after suturing together the autologous tissue.

FIGS. 14A to 14C are views showing a part of a step of the method for adjusting a breast regeneration part using a breast substitute for previous preparation, which is made of a soft resin.

FIGS. 15A and 15B are views showing a state of embedding a breast substitute or a breast substitute for previous preparation into a breast reconstruction part.

FIGS. 16A to 16C are views showing a step of creating a nipple substitute by using a nipple concave portion of the breast female mold.

### MODE FOR CARRYING OUT THE INVENTION

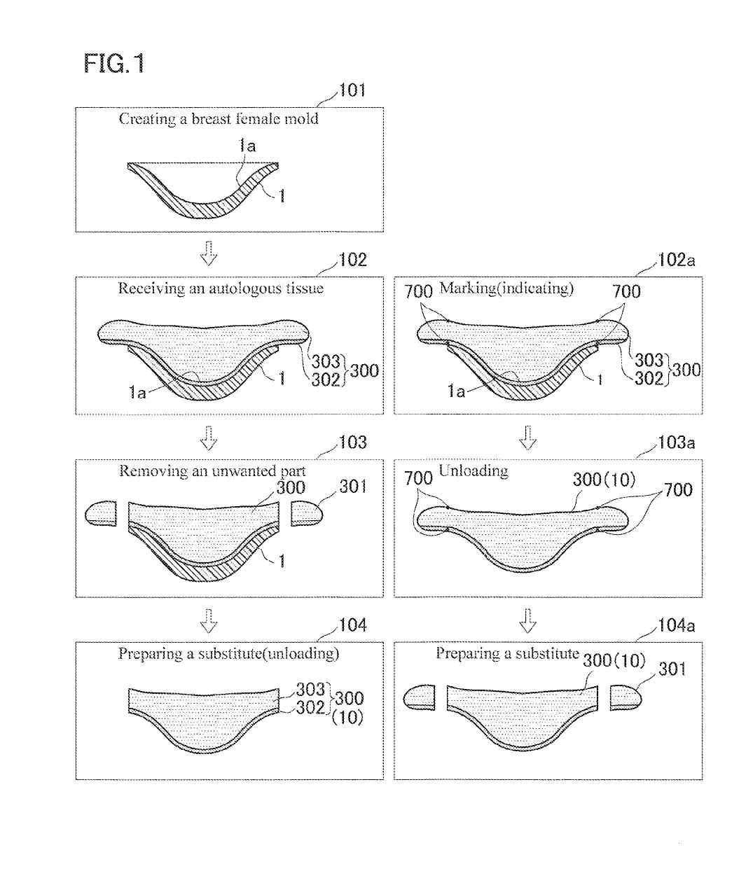

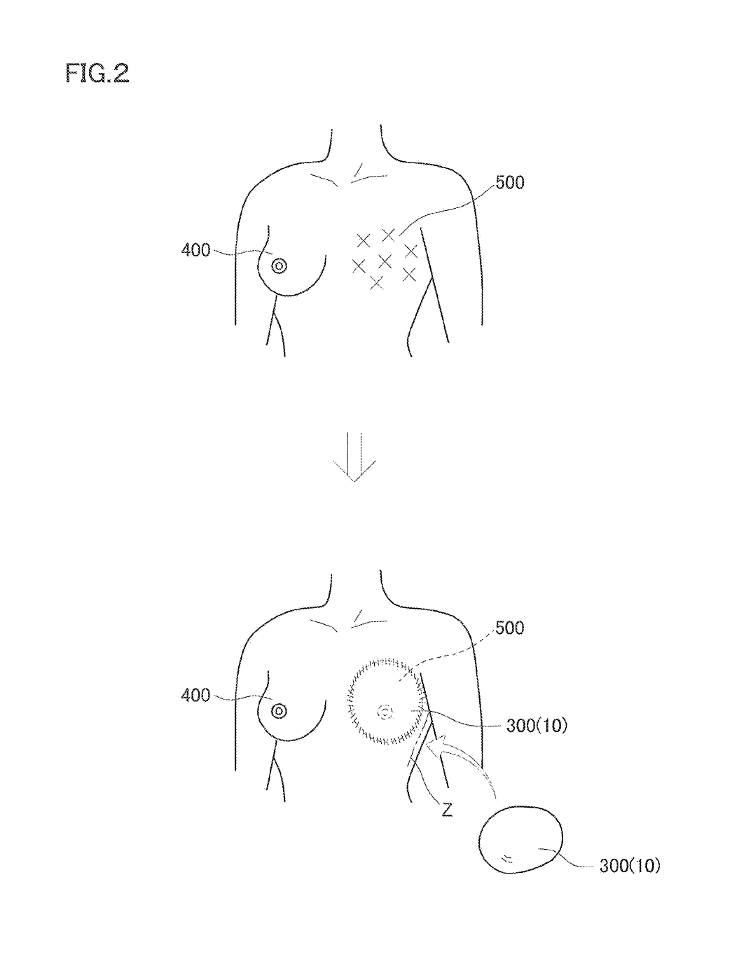

Hereinafter, embodiments of the invention will be described with reference to the drawings. FIG. 1 shows a flow chart for adjusting a breast substitute (mainly, autologous tissue) for preparation of breast reconstruction to a breast reconstruction part. FIG. 2 is a view showing a breast reconstruction part in the first reconstruction or the second reconstruction. The first reconstruction is to perform a breast reconstruction surgery in succession to a breast excision surgery, and the second reconstruction is to perform a breast reconstruction surgery after taking a suitable period from the excision of the breast.

As shown in FIGS. 1 and 2, the method for adjusting a breast regeneration part of this invention includes a breast female mold creating step 101 of creating a breast female mold 1 by transcribing the outer shape of the existing breast 400, for example, after a breast is excised due to breast cancer or the like, or when one of the breasts is congenitally defectively developed, or by transcribing the outer shape of the breast 400 before excision when the breast is excised due to breast cancer from now; and an autologous tissue receiving step 102 of receiving autologous tissues 300 in the concave portion 1a of this breast female mold 1.

In the autologous tissue receiving step 102, the autologous tissue 300 is received and filled in the concave portion 1a of the breast female mold 1 so as to bring the tissue into contact with the inner surface of the concave portion 1a and include at least an excess part in the outside (in the left and right lateral directions in the figure) of the opening of the concave portion 1a or, if necessary, an excess part in the upper side (in the upper direction in the figure).

Furthermore, the method of the invention includes, for example, the unwanted part removal step 103 of removing an excess part formed outside the concave portion 1a of the breast female mold 1 as an unwanted part 301 of the autologous tissue 300 and the substitute preparing step 104 of preparation by unloading the autologous tissue 300 from the breast female mold 1 as the breast substitute 10 after the unwanted part removal step 103. When a tissue of an excess part is removed by excision, the tissue is desirably and safely excised while watching the blood vessel in the autologous tissue 300 in order not to damage the blood vessel.

In place of removing an unwanted part 301 of the autologous tissues 300 that is excessively formed outside the concave portion 1a in the state of being received in the breast female mold 1 in the unwanted part removal step 103, the indicating step 102a of indicating (marking) a mark 700 showing the boundary of the excess part with an ink or the like may be carried out, then, the unloading step 103a of taking out the autologous tissue 300 from the breast female mold 1 may be carried out, and after unloading, the substitute preparing step 104a of preparing the breast substitute 10 may be carried out by excising the unwanted part 301 along with the mark 700.

In any case, the autologous tissue 300 for transplantation, which is a breast substitute 10 appropriately adjusted in the size as described above, is sutured to the breast defect part 500 shown in FIG. 2. For example, the breast defect part (reconstruction part) 500 is cut out from the lateral side portion (Z) of the breast defect part 500, and the breast substitute (skin flap) 300 is applied to the subcutaneous tissue of the breast defect part (reconstruction part) 500 to be sutured. Further, in order to generate a blood flow in the autologous tissue 300 for transplantation, the blood vessel of the autologous tissues 300 for transplantation and the blood vessel of the chest are sutured. Depending on an autologous tissue 300 to be selected, the autologous tissue can be transplanted in the state that the original blood vessels are continued. As described above, when a breast substitute 10 is transplanted by a surgical operation, a breast substitute 300 previously prepared and removed from the human body, which is a premise, is produced by using a breast female mold 1 that is created using the existing breast 400 or the breast before excision as a matrix for the appearance after the operation; therefore, balance of the appearance with the remaining breast 400 can be achieved.

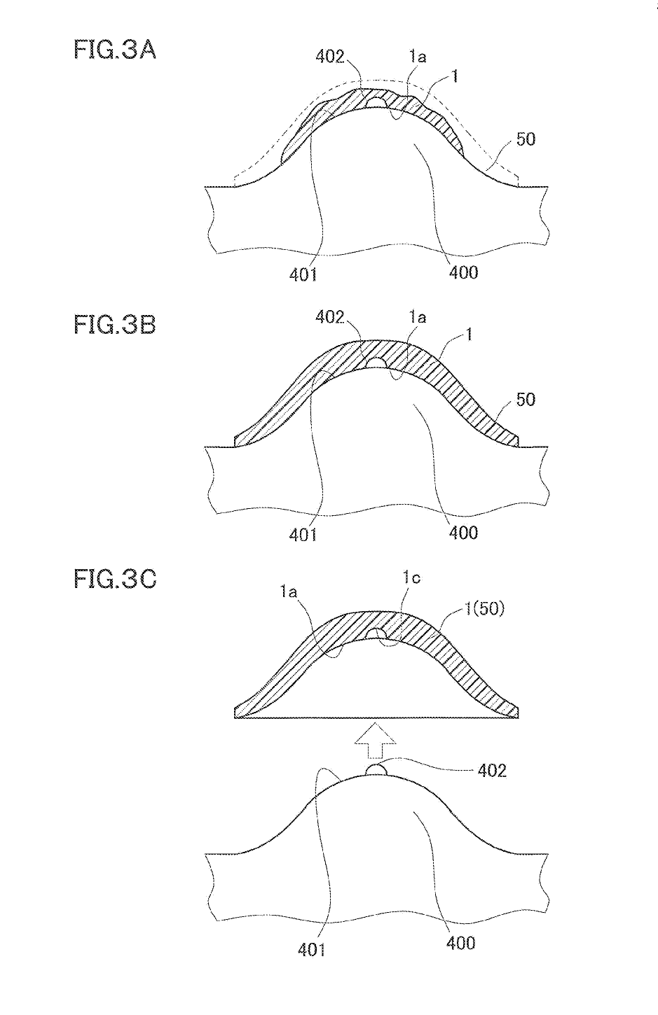

For an example of creating the breast female mold 1 in the breast female mold creating step 101, as shown in FIGS. 3A and 3B, a material obtained by mixing a silicone rubber 50 for mold making as a silicone resin, which is a synthesized resin material (resin material), as a main agent with a predetermined curing agent to be gelled is used as a material for mold making.

Then, the breast female mold creating step 101 is included as a step in which this silicone rubber 50 is built on the surface 401 of the existing breast 400 or the surface 401 of the breast 400 before excision when the breast is excised from now because of breast cancer, and a breast female mold 1 that is hardened to be in a rubbery form after taking a predetermined time for hardening is detached so as to release from the breast 400. The shape of the nipple 402 of the breast 400 is also transcribed as the nipple concave portion 1c in this breast female mold 1.

An example of this silicone rubber 50 includes a one-liquid RTV silicone rubber, other than a room temperature curing silicone rubber, a so-called two-liquid addition reaction-type RTV silicone rubber obtained by mixing a main agent and a curing agent. Examples of the silicone rubber 50 include “DENTAL ADS931A/B: manufactured by Wacker Asahikasei Silicone Co., Ltd.” and “RTV4353A/B, RTV4348A/B, RTV4358A/B, RTV4760A/B, RTV4362A/B, RTV4765A/B, RTV4388A/B: manufactured by Bluestar Silicones”. A breast female mold 1 can be obtained by hardening the silicone rubber 50 to be a rubbery format a room temperature (it is also called a normal temperature, and the temperature is prescribed at 20° C.±15° C. in the Japanese Industrial Standards (JISZ8703)).

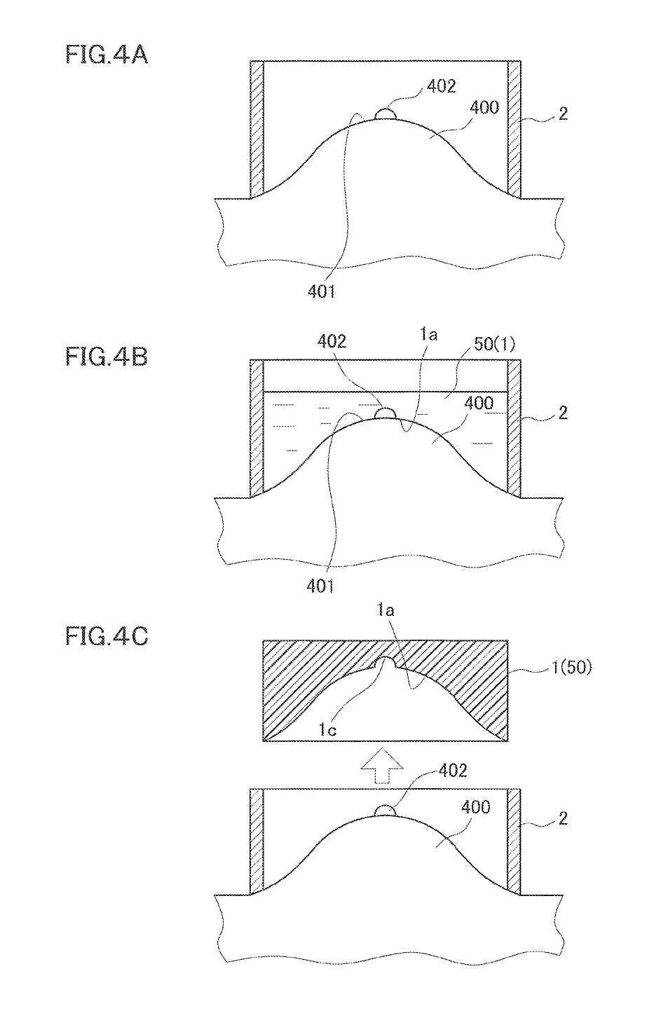

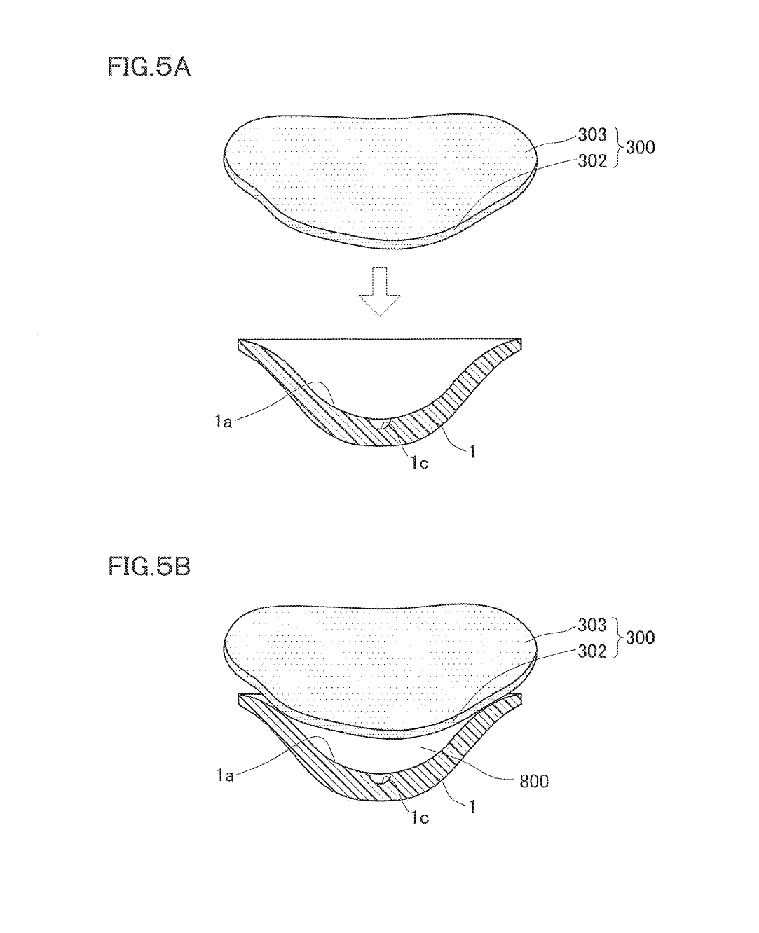

As another modified example, as shown in FIGS. 4A, 4B and 4C, the periphery of the existing breast 400 or the breast 400 before excision in the case of excising the breast because of breast cancer is enclosed with a casting mold flame 2, a silicone rubber 50 obtained by mixing a predetermined curing agent with a main agent is charged into this casting mold flame 2, and after taking a predetermined time for hardening, the breast female mold 1 hardened to be in a rubbery form can be taken out by releasing from the casting mold flame 2 and the breast 400.

For a tissue in the autologous tissues 300 in the autologous tissue receiving step 102 shown in FIG. 1, for example, a part obtained by excising from a human body with maintaining the blood flow of a human skin 302 and a subcutaneous tissue 303 is used, and this autologous tissue 300 (hereinafter also referred to as the skin flap 300) is transplanted into a breast defect part (breast reconstruction part 500) and the breast reconstruction is thus performed. In general, a latissimus dorsi skin flap or a rectus abdominis skin flap is used, and in particular, for example, a blood vessel called the deep inferior epigastric perforator (DIEP) in the lower abdomen, and skin and fat connected to the blood vessel are removed as a skin flap that is a transplanting tissue containing skin (maintained by the blood flow immediately after transplantation), or the subcutaneous fat and muscle in the back are used and they can be transplanted into the breast defect part.

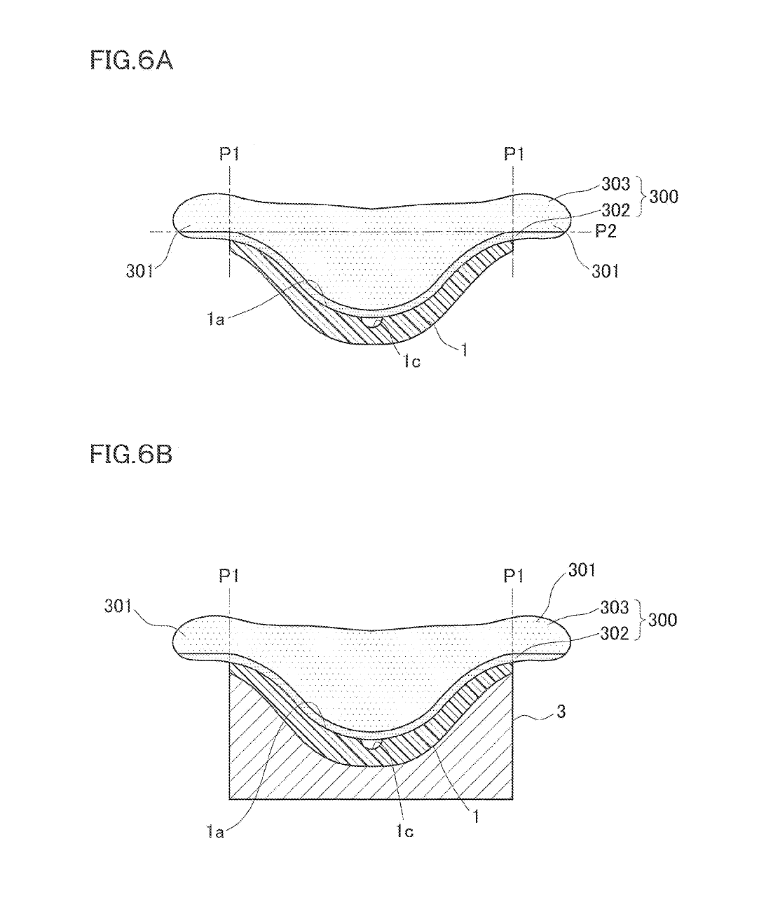

In the autologous tissue receiving step 102 in the method of the invention, as shown in FIGS. 5A and 5B, because the skin flap 300 as an autologous tissue is amorphous, the skin flap 300 is transformed so as to be embedded into the concave portion 1a in the state of receiving the skin flap 300 in the concave portion 1a of the breast female mold 1, and as shown in FIGS. 6A and 6B, the skin flap 300 is received and filled not to generate a gap 800 shown in FIG. 5B between the concave portion 1a of the breast female mold 1 and the skin flap 300 and also to include an excess part (unwanted part 301) formed outside the concave portion 1a of the breast female mold 1.

When this autologous tissue receiving step 102 is carried out, a holder base 3 (holding member) is preferably arranged in the lower side of the breast female mold 1 for suppression of transformation of the breast female mold 1 itself and position retention as shown in FIG. 6B. When this holder base 3 is mounted on the upper surface of a working table, the breast female mold 1 is kept on the holder base 3 and the skin flap 300 is received in the concave portion 1a, transformation and displacement of the breast female mold 1 can be prevented. For this holder base 3, other materials that are supposed to have higher mechanical strength than the silicone rubber 50 forming the breast female mold 1, for example, polyurethane-based resins and epoxy-based resins as harder resins, woods, ceramics, metals, and plasters can be used.

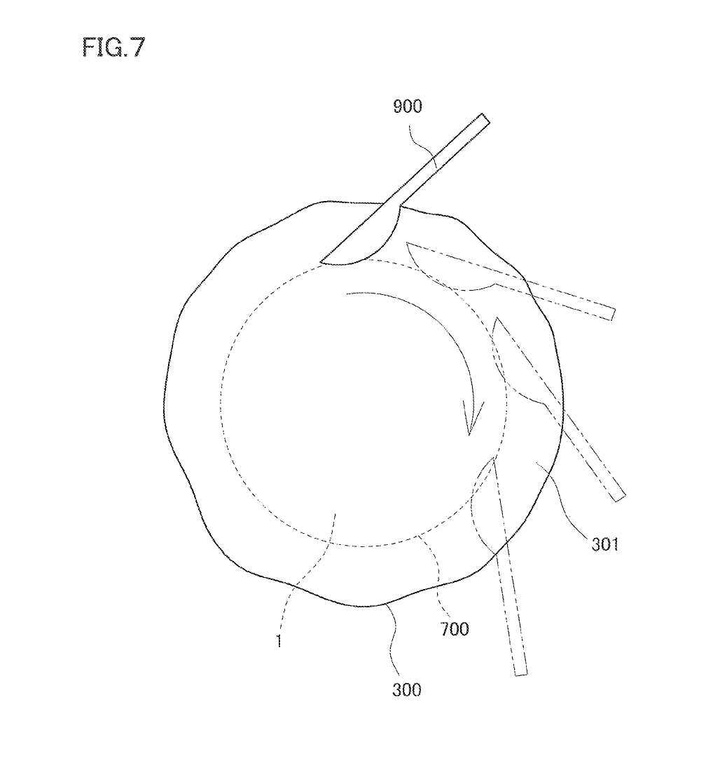

Then, in the unwanted part removal step 103 of removing an excess part formed in the outside of the periphery of the concave portion 1a of the breast female mold 1, which is shown in FIGS. 6A and 6B, as an unwanted part 301 of the skin flap 300, the unwanted part 301 of the skin flap 300, which is formed in the outside of the breast female mold 1, may be excised with a knife 900 as a surgical instrument such as scissors, surgical scissors and a surgical knife, as shown in FIG. 7.

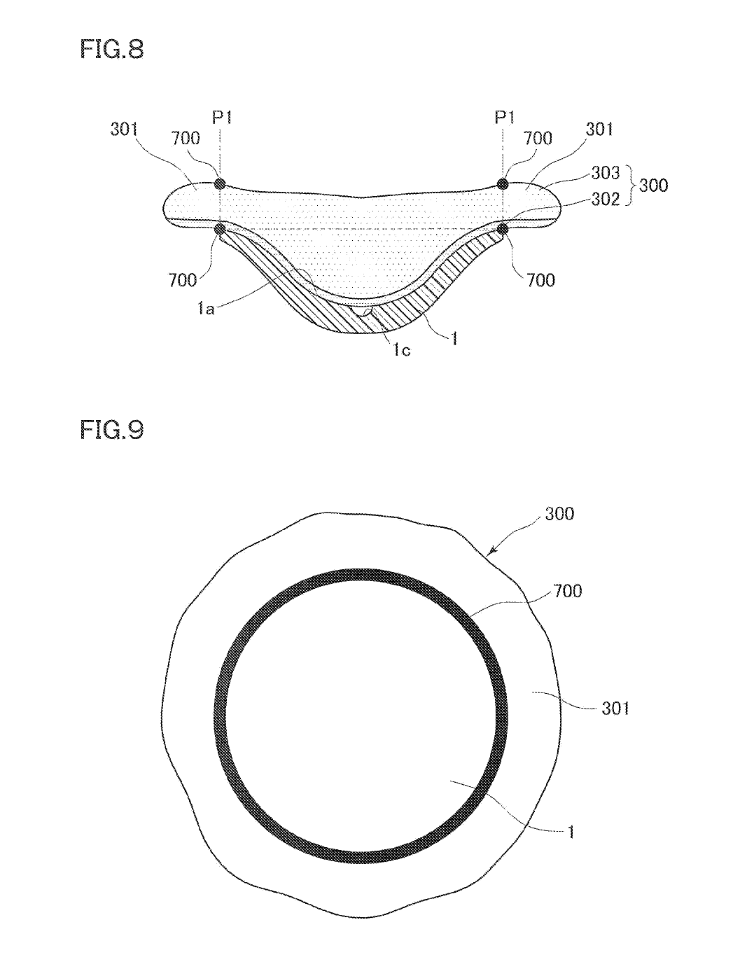

In addition, when the unwanted part removal step 103 is skipped and transferred to the indicating (marking) step 102a, as shown in FIGS. 8 and 9, a mark 700 as a boundary for removing an unwanted part 301 is indicated (marked) using, for example, an ink for a surgery. Parts indicated by the marks 700 in the indicating step 102a can be continuously or intermittently provided to at least one surface of the upper and bottom surfaces in the part P1 along with the periphery of the breast female mold 1. Then, the marks 700 in the indicating step 102a are provided to the skin flap 300 in the breast female mold 1, and the unwanted part 301 is then excised along with the marking (using as the mark) to thus form a breast substitute 10 adjusted in the size.

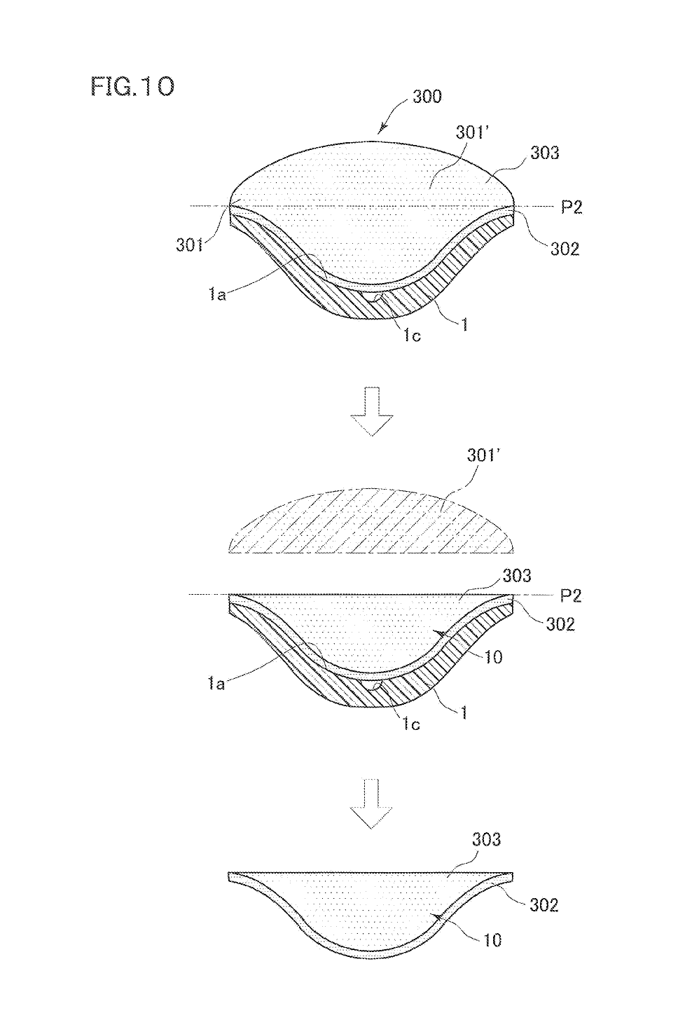

In the above described explanation, as shown in FIGS. 6A and 6B, an excess part 301 (an unwanted part) formed in the outside (the lateral side) of the peripheral edge (the opening edge of the concave portion 1a) of the breast female mold 1 is excised or the boundary is indicated by a marker or the like, and in an example of the unwanted part removal step shown in FIG. 10, an excess part formed in the upper side of the breast female mold 1 is excised. That is, an excess part 301′ (an unwanted part) formed in the upper side is excised to adjust a size generally along with the surface P2 passing through the opening upper surface of the breast female mold 1.

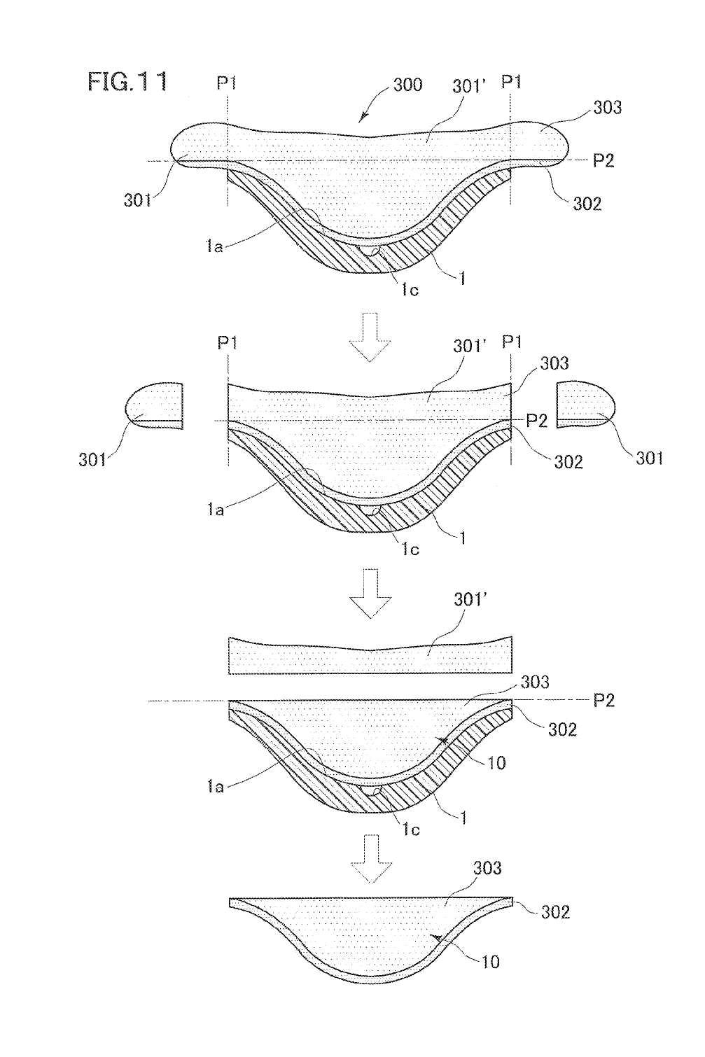

In addition, as in the unwanted part removal step shown in FIG. 11, the excess part 301 (an unwanted part) formed in the outside of the breast female mold 1 is firstly excised along with the peripheral edge P1 of the breast female mold 1, an excess part 301′ in the upper side, which is formed in the upper side of the breast female mold 1, is then excised generally along with the surface P2 passing through the opening upper surface, and a breast substitute 10 adjusted to attain a desired size may be thus obtained.

As to the order of excision, the excess part formed in the upper side may be excised and the excess part formed in the outside may be then excised. Therefore, conducting two separate excisions of the unwanted parts makes it possible to perform the unwanted part removal step precisely and easily as compared to the case of one excision.

The unwanted part removal step of excising the excess part 301′ formed in the upper side has been described in the above, and in the marker adding step (indicating step), a boundary between a necessary part and an unwanted part along with the peripheral edge of the opening upper surface in the breast female mold 1 in FIG. 10 (corresponding to the surface P2 passing through the opening upper surface) is indicated, tissues are then taken out from the breast female mold 1, and the unwanted part may be excised along with the boundary.

When boundaries with necessary parts are multiply indicated for an excess part in the outside and an excess part in the upper side of the breast female mold 1 in FIG. 11, two kinds (P1, P2) of boundaries with the excess part in the outside and the excess part in the upper side are indicated, and tissues are then taken out from the breast female mold 1 and the unwanted part removal step can be thus carried out.

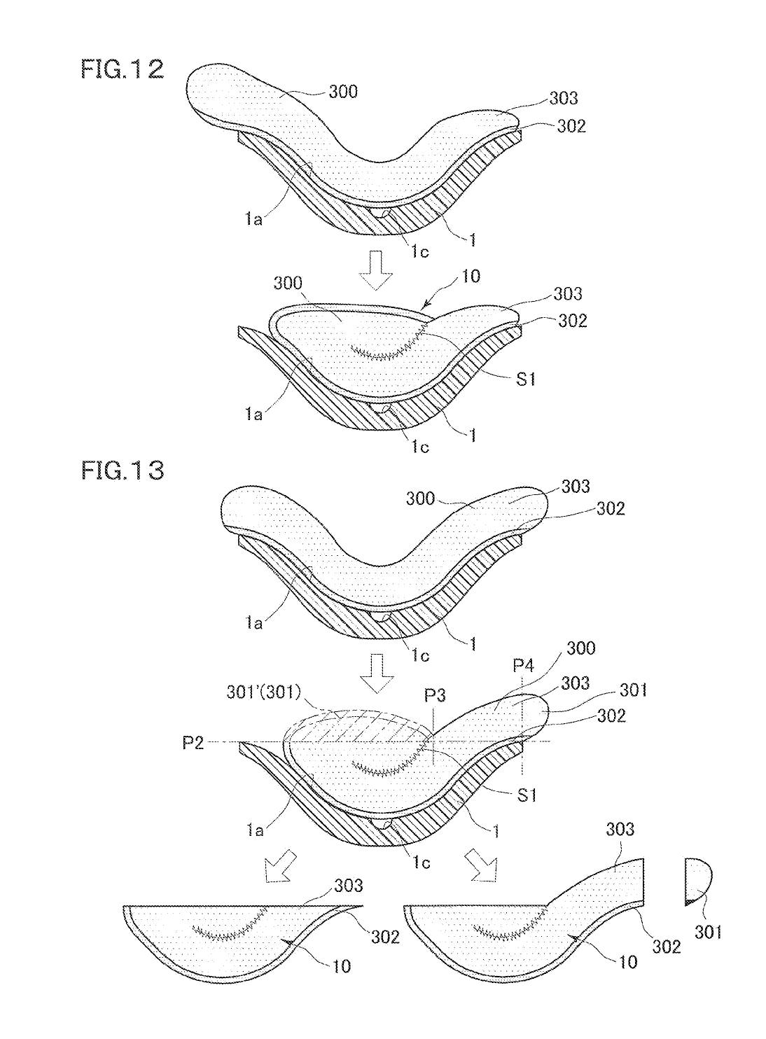

In addition, the unwanted part removal step includes the size adjusting step. As an example of creating a shape by suturing autologous tissues as the size adjusting step, as shown in FIG. 12, the skin flap 300 is received in the concave portion 1a of the breast female mold 1, a part of the skin flap 300 that is formed excessively outside the concave portion 1a is then folded back to be piled, the periphery S1 of the piled portion is sutured together and the shape of the breast substitute 10 can be thus adjusted. This example is an example of adjusting the size to conform with the breast female mold 1 by folding back and suturing excess parts formed in the outside or the upper side of the concave portion 1a of the breast female mold 1 without removing the excess parts.

As shown in FIG. 13, a part in the upper side of the opening periphery in the concave portion 1a can be removed as an unwanted part 301 from the breast substitute 10 adjusted in the shape in FIG. 12. Specifically, an excess part formed in the upper side is excised up to the position of the line P3 in the figure along with the surface P2 passing through the opening upper surface, and is excised up to the position of the line P3 in the figure along with the surface P2 passing through the opening upper surface and when the unwanted part 301 is generated in the outside, the unwanted part 301 in the outside can be excised on the line P4 in the figure (see the right figure at the bottom of FIG. 13). Furthermore, an excess part formed in the upper side can be entirely excised along with the surface P2 passing through the opening supper surface (the breast substitute 10 shown in the left figure at the bottom of FIG. 13). As a matter of course, when the boundary is indicated with a marker or the like without excision and a skin flap is taken out from the breast female mold 1, an unwanted part may be thereafter excised along with the boundary indicated with a marker or the like. In addition, as shown in FIGS. 12 and 13, as a base line for folding back and suturing an excess part of the concave portion 1a of the skin flap 300 to make the excess part smaller, for example, a suture proposition line is marked and the skin flap 300 is taken out from the breast female mold 1, and the skin flap 300 may be then sutured along with the suture proposition line to thus adjust a size.

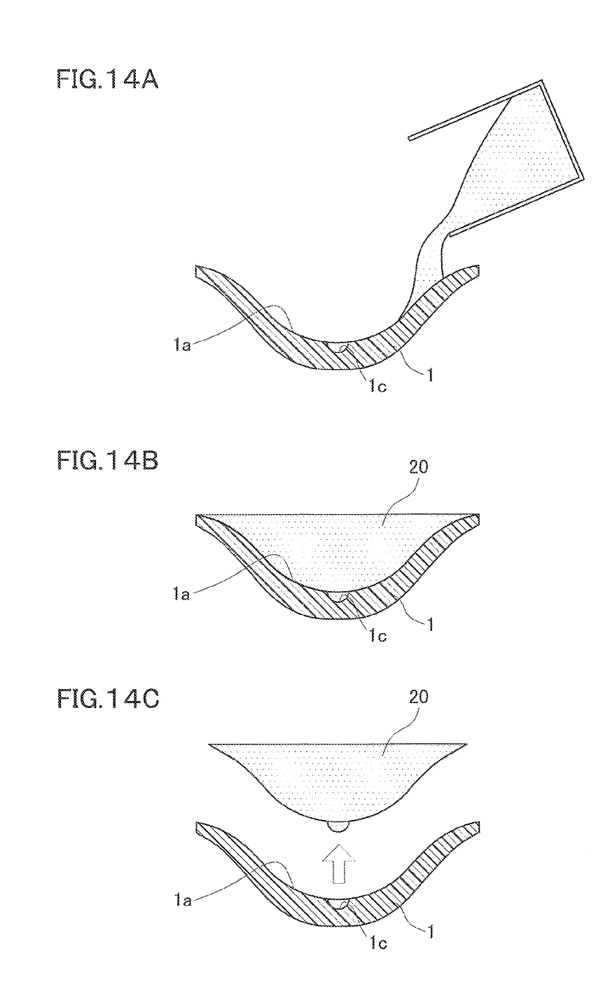

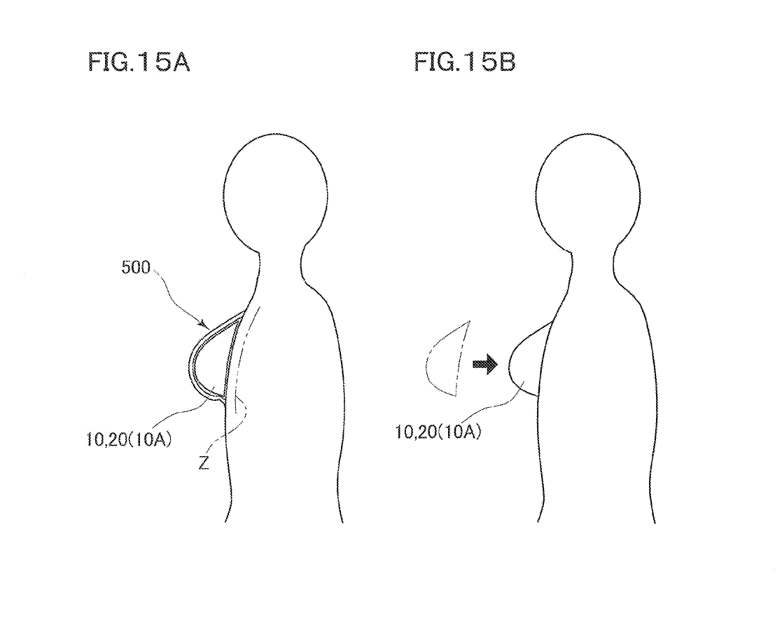

In addition, as shown in FIGS. 14A to 14C, a resin to be hardened as a soft resin such as a silicone rubber is charged or filled in the concave portion 1a of the breast female mold 1 (FIGS. 14A and 14B) and hardened, then taken out from the breast female mold 1 and a the pseudo breast model 20 made of a soft resin (for example, made of a silicone rubber) can be thus created. Preparatory for subcutaneously applying the breast substitute 10 (skin flap 300) to the breast reconstruction part 500 and suturing as shown in FIG. 2, the above described pseudo breast model 20 can be subcutaneously applied to the breast defect part 500 to confirm a suture position (a position of suturing the breast substitute 10 (skin flap 300) is determined at a position capable of achieving balance between the left and right breasts for the existing breast, and so on), as shown in FIG. 15A. In this case, for example, apart of the chest skin in the breast defect part 500 is cut open (Z) from the underarm, the pseudo breast model 20 is temporarily inserted first and applied from the dissected part (Z), a suture position of the breast substitute 10 (skin flap 300) that is inserted later is then confirmed and predetermined, thereafter the pseudo breast model 20 is taken out from the subcutaneous, the original breast substitute 10 (skin flap 300) is inserted under the skin in place of the pseudo breast model 20, and the skin flap 300 is sutured to a chest subcutaneous tissue at the above described suture position that is previously confirmed and predetermined, and the dissected part Z in the underarm is also sutured to be closed. As shown in FIG. 15B, the pseudo breast model 20 may be used so as to apply to the breast defect part from the outside.

Further, in the unwanted part removal step 103 and the marking step 102a in FIG. 1, the size adjusting step or the like in FIGS. 12 and 13, the size of the breast substitute 10 can be precisely and easily adjusted by carrying out the above described steps with reference to this pseudo breast model 20.

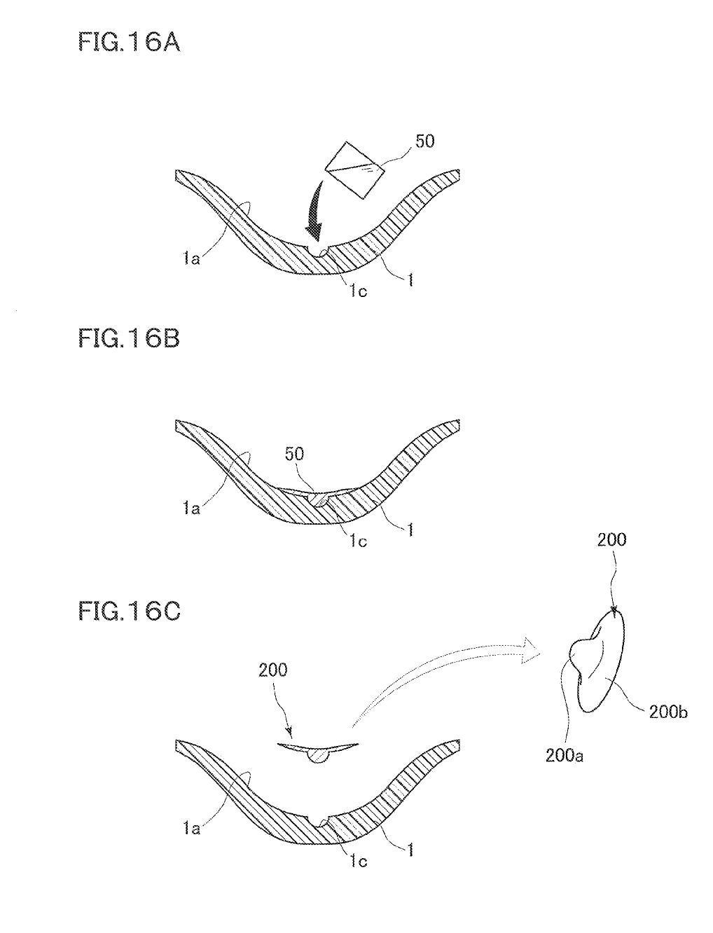

As shown in FIGS. 3A to 3C and 4A to 4C, when the existing breast 400 is transcribed into the breast female mold 1, the nipple part of the existing breast 400 (the nipple and the areola mammae around the nipple) is also transcribed into the concave portion 1a of the breast female mold 1 as the nipple concave portion 1c. This nipple concave portion 1c is not directly involved with a function of adjusting the size of the skin flap 300 that is obtained for breast reconstruction; however, even if the skin flap 300 is transplanted into the chest, it becomes a swollen form without a nipple part, and therefore, a nipple substitute 200 artificially created on the top of this swollen surface, which is shown in FIGS. 16A to 16C, can be adhered to the breast reconstruction part 500 shown in FIG. 2.

In order to produce this nipple substitute 200 made of, for example, a silicone rubber, the transcribed portion (the concave 1c of the concave portion 1a) to the concave portion 1a of the remaining nipple portion is used. That is, as shown in FIGS. 16A to 16C, the nipple substitute 200 including the nipple 200a and the areola mammae 200b is produced using the nipple concave portion 1c formed in the concave portion 1a of the breast female mold 1. A similar silicone rubber 50 as used in the breast female mold 1 is poured into this nipple concave portion 1c and hardened to be formed, this nipple substitute 200 is released from the mold, and then the nipple substitute 200 can be adhered to the breast substitute 10.

As described above, the embodiments of the invention were explained; however, these embodiments are merely examples, the invention is not limited thereto, and in each embodiment, a constitution described as a part of an embodiment for explanation of the invention can be utilized in another embodiment, and these can be combined to also form other embodiments.

### EXPLANATION OF SYMBOLS

1 Breast female mold
1a Concave portion
10 Breast substitute
101 Breast female mold creating step
102 Autologous tissues receiving step
102a Marking (indicating) step
103 Unwanted part removal step
103a Unloading step
104, 104a Substitute preparing step
300 Autologous tissues (skin flap)
301, 301′ Unwanted parts

1. A method for adjusting a breast regeneration part, comprising, when adjusting a breast substitute for the preparation of breast reconstruction; a breast female mold creating step of transcribing an existing breast or a breast to be removed and creating a breast female mold;
a receiving step of receiving the breast substitute in the concave portion of the breast female mold so as to include an excess part of the concave portion;
a size adjusting step of removing the excess part of the concave portion of the breast substitute as an unwanted part of the breast substitute or folding back the excess part so that an excess part does not form, or an indicating step of indicating the boundary for removing the excess part of the concave portion of the breast substitute as an unwanted part of the breast substitute or indicating a base line for folding back the excess part so that an excess part does not form; and
a substitute preparing step of unloading from the breast female mold and preparing as the breast substitute after the size adjusting step or the indicating step.

2. The method for adjusting a breast regeneration part according to claim 1, wherein the size adjusting step is an unwanted part removal step of removing the excess part of the concave portion of the breast female mold.

3. The method for adjusting a breast regeneration part according to claim 1, wherein the excess part of the concave portion of the breast female mold comprises at least one of an excess part in the outside of the concave portion and an excess part in the upper side.

4. The method for adjusting a breast regeneration part according to claim 1, wherein the size adjusting step is a step of folding back and suturing the excess part of the concave portion of the breast female mold to reduce the size.

5. The method for adjusting a breast regeneration part according to claim 1, wherein the breast female mold creating step comprises a step of building a gelled resin material on the surface of the existing breast or the surface of the breast to be removed, hardening the resin material, and then unloading the resin material from the breast.

6. The method for adjusting a breast regeneration part according to claim 1, wherein the breast substitute is a living tissue and sutured to a breast reconstruction part.

7. The method for adjusting a breast regeneration part according to claim 1, comprising a step of creating a pseudo breast model made of a soft resin after creating the breast female mold, by injecting or filling a fluid resin into the concave portion of the breast female mold to be hardened before or after creation of the breast substitute and then unloading the resin from the breast female mold,
wherein the pseudo breast model obtained in the step is referred in the size adjusting step or the indicating step, or used in confirmation of a suture position of the breast substitute to a breast reconstruction part.
